(19) United States
(12) Patent Application Publication
(10) Pub. No.: US 2010/0087499 A1
(43) Pub. Date: Apr. 8, 2010

(54) PHARMACEUTICAL COMPOSITIONS AND METHODS OF USING TEMOZOLOMIDE AND MULTI-TARGETED KINASE INHIBITORS

(75) Inventors: Yaolin Wang, Short Hills, NJ (US); Ming Liu, Fanwood, NJ (US); Walter Robert Bishop, Pompton Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION, Kenilworth, NJ (US)

(21) Appl. No.: 12/523,809

(22) PCT Filed: Jan. 28, 2008
(86) PCT No.: PCT/US2008/001061

§ 371 Date: Nov. 24, 2009

Related U.S. Application Data
(60) Provisional application No. 60/887,245, filed on Jan. 30, 2007.

(51) Int. Cl. A61K 31/4188; A61P 35/00

(52) U.S. Cl. 514/393

(57) ABSTRACT

The present invention provides formulations, kits and methods useful for treating cell proliferative disorder. In particular, the formulations, kits and methods include temozolomide (TMZ) in combination with a multi-targeted kinase inhibitor.

### FIELD OF THE INVENTION

The present invention provides formulations, kits, and methods for treating a cell proliferative disorder. In particular, the formulations, kits and methods include temozolomide (TMZ) and a multi-targeted kinase inhibitor.

### BACKGROUND OF THE INVENTION

In light of the widespread number of cancer cases and cancer-related deaths, as well as the inadequacies of currently available treatments, there is a need for more effective therapeutics to treat cancers. Such cancers include glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides.

Of all brain tumors diagnosed each year in the United States, about half are malignant gliomas and result in death within 18 months. Gliomas originate from glial cells, most often astrocytes, and may occur anywhere in the brain or spinal cord, including the cerebellum, brain stem, or optic chiasm. Gliomas can be divided into two groups based on their growth characteristics: low-grade gliomas and high-grade gliomas. Low-grade gliomas are usually localized and grow slowly over a long period of time. Examples of low-grade gliomas include astrocytomas, oligodendrogliomas, pilocytic astrocytomas. Over time, most of these low-grade gliomas dedifferentiate into more malignant high-grade gliomas that grow rapidly and can easily spread through the brain. Examples of high-grade gliomas include anaplastic astrocytoma and glioblastoma multiforme.

Despite advances in conventional therapies for malignant gliomas which include surgical removal, radiation therapy, and chemotherapy as well as combinations thereof, malignant gliomas continue to be associated with a poor prognosis. Thus, there remains a need for more effective therapeutics to treat the growth and metastasis of a variety of cancers, including gliomas.

Cancer results from a defect in the regulation of processes that control cell proliferation and survival. Kinases are a large set of enzymes that transmit signals to the cell's nucleus to control biological processes such as growth and differentiation of cells. In many cancer cells, the process of growth and differentiation is disregulated. This disregulation may be the result of one or more protein kinases being continually “on”.

The use of multi-targeted kinase inhibitors for cancer therapy are attractive because one agent or compound can inhibit multiple kinases. Examples of multi-targeted kinase inhibitors include Sutent® (sunitinib; SU11248) from Pfizer, Nexavar® (sorafenib; Bay 43-9006) from Onyx Pharmaceuticals; Sprycel™ (dasatinib; BMS-354825) from Bristol-Myers Squibb; Zactima® (ZD6474) from AstraZeneca; Tykerb® (lapatinib) from Glaxo Smith Kline; STI571 from Novartis; AMG 706 from Amgen; MP-412 from Aveo Pharmaceuticals; CEP-701 (lestaurtinib) from Cephalon; XL647 from Exelixis; XL999 from Exelixis; MLN518 (formerly known as CT53518) from Millennium Pharmaceuticals; PKC412 from Novartis; AMN 107 from Novartis; AEE 788 from Novartis; OSI-930 from OSI Pharmaceuticals; OSI-817 from OSI Pharmaceuticals; axitinib (AG-013736) from Pfizer; ARRY-334543 from Array BioPharma, MG-90265 from MethylGene, Inc and AZD6244 (ARRY-142886). See Branca et al., “Multi-Target Kinase Inhibitors Hitting the Market”, PharmaWeek, Feb. 9, 2006.

TMZ is an alkylating agent available under the trademark Temodar® from Schering Corporation (Kenilworth, N.J.). TMZ is also known as 3,4-dihydro-3-methyl-4-oxoimidazo[5,1-d]-as-tetazine-8-carboxamide. See U.S. Pat. No. 5,260,291, incorporated herein by reference in its entirety. TMZ is currently approved in the United States for the treatment of adult patients with high grade gliomas that include newly diagnosed glioblastoma multiforme and refractory anaplastic astrocytoma. TMZ is also approved in other countries for the treatment of malignant gliomas and for the treatment of melanomas.

### SUMMARY OF THE INVENTION

The present invention provides formulations, kits, and methods useful for treating a cell proliferative disorder.

In some embodiments, the invention encompasses methods, formulations and kits for treating a patient having a cell proliferative disorder comprising administering to the patient therapeutically effective amounts of an alkylating agent and a multi-targeted kinase inhibitor.

In other embodiments, the invention encompasses methods, formulations and kits for treating a patient having a cell proliferative disorder comprising administering to the patient therapeutically effective amounts of a chemotherapeutic agent and a multi-targeted kinase inhibitor.

In other embodiments, the invention encompasses methods, formulations and kits for treating a patient having a cell proliferative disorder comprising administering to the patient therapeutically effective amounts of an alkylating agent and an angiogenesis inhibitor.

In other embodiments, the invention encompasses methods, formulations and kits for treating a patient having a cell proliferative disorder comprising administering to the patient therapeutically effective amounts of a chemotherapeutic agent and an angiogenesis inhibitor.

The alkylating agent could be any alkylating agent (including nitrogen mustards, ethylenimine derivatives, alkyl sulfonates, nitrosoureas and triazenes). Non-limiting examples of alkylating agents include: uracil mustards, chlormethine, cyclophosphamide (Cytotaxan®), ifosfamide, melphalan, chlorambucil, pipobroman, triethylene-melamine, triethylenethiophosphoramine, busulfan, carmustine, lomustine, streptozaocin, decarbazine, and temozolomide (TMZ). In a preferred embodiment, the alkylating agent is TMZ.

In certain embodiments, the present invention provides formulations, kits, and methods that include TMZ, or a pharmaceutically acceptable salt thereof, in combination with a multi-targeted kinase inhibitor. Such a combination is more effective than treatment with either therapy alone. In addition, the present formulations, kits, and methods permit a lower dose of one or more pharmaceutically active agents to be administered, than would otherwise be required, to achieve a therapeutic effect thereby reducing adverse effects associated with the dosage administered.

The cell proliferative disorder can be any cell proliferative disorder. In preferred embodiments, the cell proliferative disorder is glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides. In other preferred embodiments, the cell proliferative disorder is glioma, melanoma, lung cancer, lymphoma, colorectal and/or colon cancer, head and neck or ovarian cancer. In a preferred embodiment, the cell proliferative disorder is glioma. In another preferred embodiment, the cell proliferative disorder is melanoma.

The multi-targeted kinase inhibitor could be any multi-targeted kinase inhibitor. In certain embodiments, the multi-targeted kinase inhibitor is selected from the group consisting of: sunitinib, sorafenib, dasatinib, Zactima®, lapatinib, STI571, AMG 706, MP-412, CEP-701, XL647, XL999, MLN518, PKC412, AMN107, AEE 788, OSI-930, OSI-817, axitinib (AG-013736), ARRY-334543, MG-90265 and AZD6244 (ARRY-142886); or pharmaceutically acceptable salts thereof, or a combination of two or more thereof. In a preferred embodiment, the multi-targeted kinase inhibitor is sunitinib or a pharmaceutically acceptable salt thereof (including, but not limited, to sunitinib malate). In a preferred embodiment, the multi-targeted kinase inhibitor is sorafenib or a pharmaceutically acceptable salt thereof (including, but not limited, to sorafenib tosylate).

In preferred embodiments, the present invention provides methods for treating a patient having a cell proliferative disorder comprising administering to the patient a therapeutically effective amount of TMZ (or a pharmaceutically acceptable salt thereof) and a therapeutically effective amount of a multi-targeted kinase inhibitor. In one preferred embodiment of the method, the cell proliferative disorder is a brain tumor. In one embodiment, the brain tumor is a glioma. In one embodiment, the glioma is an anaplastic astrocytoma. In another preferred embodiment, the glioma is a glioblastoma multiforme.

The multi-targeted kinase inhibitor could be any multi-targeted kinase inhibitor, or a combination of two or more multi-targeted kinase inhibitors. In certain embodiments, the multi-targeted kinase inhibitor is selected from the group consisting of: sunitinib, sorafenib, dasatinib, Zactima®, lapatinib, STI571, AMG 706, MP-412, CEP-701, XL647, XL999, MLN518, PKC412, AMN107, AEE 788, OSI-930, OSI-817, axitinib (AG-013736), ARRY-334543, MG-90265 and AZD6244 (ARRY-142886); or pharmaceutically acceptable salts thereof, or a combination of two or more thereof. In a preferred embodiment of the method, the multi-targeted kinase inhibitor is sunitinib, or a pharmaceutically acceptable salt thereof (for example, sunitinib malate, which is currently marketed under the trademark Sutent®). Sunitinib is described in U.S. Pat. Nos. 6,573,293 and 7,125,905. In another embodiment of the method, the multi-targeted kinase inhibitor is sorafenib or a pharmaceutically acceptable salt thereof (for example sorafenib tosylate, which is currently marketed under the trademark Nexavar®). In another embodiment of the method, the multi-targeted kinase inhibitor is. In another embodiment of the method, the multi-targeted kinase inhibitor is AZD6244 (ARRY-142886) or a pharmaceutically acceptable salt thereof. AZD6244 (ARRY-14266) is described, e.g., in Clin. Cancer Res. 13(5):1576-83 (2007).

In one embodiment, wherein the cell proliferative disorder being treated is melanoma, the multi-targeted kinase inhibitor is not sorafenib or a pharmaceutically acceptable salt thereof.

In certain embodiments of the method, the invention provides methods for treating a patient having a brain tumor comprising administering to the patient a therapeutically effective amount of temozolomide or a pharmaceutically acceptable salt thereof and a therapeutically effective amount of sunitinib or a pharmaceutically acceptable salt thereof. In one embodiment the brain tumor is a glioma. In one embodiment, the brain tumor is a glioma. In one embodiment, the glioma is an anaplastic astrocytoma. In another preferred embodiment, the glioma is a glioblastoma multiforme.

In other embodiments of the method, the invention provides methods for treating a patient having melanoma comprising administering to the patient a therapeutically effective amount of temozolomide or a pharmaceutically acceptable salt thereof and a therapeutically effective amount of sunitinib or a pharmaceutically acceptable salt thereof.

In other embodiments of the method, the pharmaceutically acceptable salt of TMZ or of the multi-targeted kinase inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In certain embodiments of the method, the therapeutically effective amount of TMZ (or a pharmaceutically acceptable salt thereof) ranges from about 75 mg/m2 of BSA per day to about 450 mg/m2 of BSA per day. In preferred embodiments of the method, the therapeutically effective amount of TMZ (or pharmaceutically acceptable salt thereof) ranges from about 75 mg/m2 of BSA per day to about 250 mg/m2 of BSA per day. In other preferred embodiments of the method, the therapeutically effective amount of TMZ (or pharmaceutically acceptable salt thereof) is 75 mg/m2, 100 mg/m2, 150 mg/m2 or 200 mg/m2 of BSA per day.

In certain embodiments of the method, the therapeutically effective amount of TMZ (or a pharmaceutically acceptable salt thereof) is a dose of TMZ adequate to achieve a standard dose intensity of TMZ. In another embodiment of the method, the therapeutically effective amount of TMZ (or a pharmaceutically acceptable salt thereof) is a dose of TMZ adequate to achieve an enhanced dose intensity of TMZ.

In certain embodiments of the method, the therapeutically effective amount of sunitinib or a pharmaceutically acceptable salt thereof is about 50 mg per day. In one embodiment, sunitinib or a pharmaceutically acceptable salt thereof is administered at about 50 mg per day for 4 weeks, followed by a rest period of two weeks.

In other embodiments of the method, the therapeutically effective amount of sorafenib or a pharmaceutically acceptable salt thereof is about 800 mg per day.

In some embodiments of the method, the TMZ (or a pharmaceutically acceptable salt thereof) and the multi-targeted kinase inhibitor are administered at the same time. In other embodiments, TMZ (or a pharmaceutically acceptable salt thereof) and the multi-targeted kinase inhibitor are administered at different times. Thus, for example, the TMZ or a pharmaceutically acceptable salt thereof and the multi-targeted kinase inhibitor may be administered on the same days or on different days, and/or at the same time or at different times.

Further, the TMZ (or a pharmaceutically acceptable salt thereof) and the multi-targeted kinase inhibitor may be administered in combination with any other treatment and/or chemotherapeutic agent. In certain embodiments, the TMZ or a pharmaceutically acceptable salt thereof and the multi-targeted kinase inhibitor may be administered before and/or after surgery. In other embodiments, the TMZ or a pharmaceutically acceptable salt thereof and the multi-targeted kinase inhibitor may be administered before, during or after radiation treatment.

In preferred embodiments, the present invention provides formulations comprising a therapeutically effective amount of TMZ (or a pharmaceutically acceptable salt thereof) and a therapeutically effective amount of a multi-targeted kinase inhibitor. The multi-targeted kinase inhibitor could be any multi-targeted kinase inhibitor, or a combination of two or more multi-targeted kinase inhibitors. In certain embodiments, the multi-targeted kinase inhibitor is selected from the group consisting of: sunitinib, sorafenib, dasatinib, Zactima®, lapatinib, STI571, AMG 706, MP-412, CEP-701, XL647, XL999, MLN518, PKC412, AMN107, AEE 788, OSI-930, OSI-817, axitinib (AG-013736), ARRY-334543, MG-90265 and AZD6244 (ARRY-142886); or pharmaceutically acceptable salts thereof, or a combination of two or more thereof. In a one preferred embodiment of the formulation, the multi-targeted kinase inhibitor is sunitinib, or a pharmaceutically acceptable salt thereof (for example, sunitinib malate). In another embodiment of the formulation, the multi-targeted kinase inhibitor is sorafenib or a pharmaceutically acceptable salt thereof (for example, sorafenib tosylate).

In certain embodiments of the formulation, the pharmaceutically acceptable salt of TMZ or the multi-targeted kinase inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In certain embodiments of the formulation, the therapeutically effective amount of TMZ (or a pharmaceutically acceptable salt thereof) ranges from about 75 mg/m2 of BSA per day to about 450 mg/m2 of BSA per day. In preferred embodiments of the formulation, the therapeutically effective amount of TMZ (or pharmaceutically acceptable salt thereof) ranges from about 75 mg/m2 of BSA per day to about 250 mg/m2 of BSA per day. In other preferred embodiments of the formulation, the therapeutically effective amount of TMZ (or pharmaceutically acceptable salt thereof) is 75 mg/m2, 100 mg/m2, 150 mg/m2 or 200 mg/m2 of BSA per day.

In certain embodiments of the formulation, the therapeutically effective amount of TMZ (or a pharmaceutically acceptable salt thereof) is a dose of TMZ adequate to achieve a standard dose intensity of TMZ. In another embodiment of the formulation, the therapeutically effective amount of TMZ (or a pharmaceutically acceptable salt thereof) is a dose of TMZ adequate to achieve an enhanced dose intensity of TMZ.

In certain embodiments of the formulation, the therapeutically effective amount of sunitinib, or a pharmaceutically acceptable salt thereof is about 50 mg per day. In one embodiment, sunitinib or a pharmaceutically acceptable salt thereof is administered at about 50 mg per day for 4 weeks, followed by a rest period of two weeks.

In other embodiments of the claimed formulation, the therapeutically effective amount of sorafenib or a pharmaceutically acceptable salt thereof is about 800 mg per day.

In preferred embodiments, the present invention also provides kits comprising:
a) a first container having a therapeutically effective amount of TMZ or a pharmaceutically acceptable salt thereof;
b) a second container having a therapeutically effective amount of a multi-targeted kinase inhibitor; and
c) instructions for use to treat a cell proliferative disorder.

In certain embodiments the cell proliferative disorder is a brain tumor. In some embodiments, the brain tumor is a glioma. In other embodiments, the glioma is an anaplastic astrocytoma. In other embodiments, the glioma is a glioblastoma multiforme.

In other embodiments, the cell proliferative disorder is melanoma.

The multi-targeted kinase inhibitor could be any multi-targeted kinase inhibitor, or a combination of two or more multi-targeted kinase inhibitors. In certain embodiments, the multi-targeted kinase inhibitor is selected from the group consisting of: sunitinib, sorafenib, dasatinib, Zactima®, lapatinib, STI571, AMG 706, MP-412, CEP-701, XL647, XL999, MLN518, PKC412, AMN107, AEE 788, OSI-930, OSI-817, axitinib (AG-013736), ARRY-334543, MG-90265 and AZD6244 (ARRY-142886); or pharmaceutically acceptable salt thereof, or a combination of two or more thereof. In a preferred embodiment of the kit, the multi-targeted kinase inhibitor is sunitinib, or a pharmaceutically acceptable salts thereof (for example, sunitinib malate). In another embodiment of the kit, the multi-targeted kinase inhibitor is sorafenib or a pharmaceutically acceptable salt thereof (for example, sorafenib tosylate).

In certain embodiments of the kit, the pharmaceutically acceptable salt of TMZ or of the multi-targeted kinase inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In certain embodiments of the kit, the therapeutically effective amount of TMZ (or a pharmaceutically acceptable salt thereof) ranges from about 75 mg/m2 of BSA per day to about 450 mg/m2 of BSA per day. In preferred embodiments of the kit, the therapeutically effective amount of TMZ (or pharmaceutically acceptable salt thereof) ranges from about 75 mg/m2 of BSA per day to about 250 mg/m2 of BSA per day. In other preferred embodiments of the kit, the therapeutically effective amount of TMZ (or pharmaceutically acceptable salt thereof) is 75 mg/m2, 100 mg/m2, 150 mg/m2 or 200 mg/m2 of BSA per day.

In other embodiments of the kit, the therapeutically effective amount of TMZ (or a pharmaceutically acceptable salt thereof) is a dose of TMZ adequate to achieve a standard dose intensity of TMZ. In other embodiments of the kit, the therapeutically effective amount of TMZ (or a pharmaceutically acceptable salt thereof) is a dose of TMZ adequate to achieve an enhanced dose intensity of TMZ.

In certain embodiments of the kit, the therapeutically effective amount of sunitinib or a pharmaceutically acceptable salt thereof is about 50 mg per day.

In other embodiments of the claimed kit, the therapeutically effective amount of sorafenib or a pharmaceutically acceptable salt thereof is about 800 mg per day.

In certain embodiments of the kit, the instructions for use recite a dosing regiment of TMZ adequate to achieve a standard dose intensity of TMZ.

In certain embodiments of the kit, the instructions for use recite a dosing regiment of TMZ adequate to achieve an enhanced dose intensity of TMZ.

In certain embodiments of the kit, the TMZ (or a pharmaceutically acceptable salt thereof) and the multi-targeted kinase inhibitor are administered at the same time. In another preferred embodiment of the kit, the therapeutically effective amount of TMZ (or a pharmaceutically acceptable salt thereof) and the multi-targeted kinase inhibitor are administered at different times.

### BRIEF DESCRIPTION OF THE DRAWINGS

FIG. 1 illustrates mean tumor growth curves of U87MG (glioblastoma) xenograft tumors in nude mice dosed with control or various amounts of TMZ, sunitinib, or a combination of both TMZ and sunitinib. (See Table 1 for dosing regimens.)

FIG. 2 illustrates U87MG (glioblastoma) xenograft mean tumor size (mm³), % tumor inhibition, and % tumor regression on Day 17 (post inoculation) after dosing with control or various amounts of TMZ, sunitinib, or a combination of both TMZ and sunitinib. (See Table 1 for dosing regimens.)

FIG. 3 illustrates mean tumor growth curves of U87MG (glioblastoma) xenograft tumors in nude mice dosed with control or various amounts of TMZ, sunitinib, or a combination of both TMZ and sunitinib. (See Table 2 for dosing regimens.)

FIG. 4 illustrates U87MG (glioblastoma) xenograft mean tumor size (mm³), % tumor inhibition, and % tumor regression on Day 20 (post inoculation) in nude mice after dosing with control or various amounts of TMZ, sunitinib, or a combination of both TMZ and sunitinib. (See Table 2 for dosing regimens.)

### DETAILED DESCRIPTION OF THE INVENTION

As used herein, the following terms shall have the definitions set forth below.

As used herein, the phrase “multi-targeted kinase inhibitor” refers to a compound or agent that inhibits the biological activity of two or more kinases. Non-limiting examples of kinases that may be inhibited by the multi-targeted kinase inhibitor include, without limitation, tyrosine kinases and serine/threonine kinases. In one embodiment, the multi-targeted kinase inhibitor inhibits more than one tyrosine kinase, for example, one or more kinases selected from the group consisting of: the tyrosine kinases c-Src, c-Abl, cKIT, BCR-ABL, Flt-3, EGFR (epidermal growth factor receptor(s)), VEGFR (vascular endothelial growth factor receptor(s)), HER2 (human epidermal growth factor receptor(s)), PDGFR (platelet-derived growth factor receptor(s)); the serine/threonine kinases B-Raf, MEK, ERK, AKT, mTOR, PDK1; or the lipid kinase PI3K. Methods of determining whether a compound or agent inhibits the biological activity of a kinase are well known in the art.

As used herein, the phrase “therapeutically effective amount” with respect to TMZ or a multi-targeted kinase inhibitor means an amount which provides a therapeutic benefit in the treatment or management of a cell proliferative disorder (e.g., glioma, etc.). In preferred embodiments, the therapeutically effective amount of temozolomide or of the multi-targeted kinase inhibitor is less that would be required by either therapy alone to achieve a therapeutic effect thereby reducing adverse effects associated with the dosage administered.

As used herein the phrase “pharmaceutically acceptable salt” refers to a non-toxic salt prepared from a pharmaceutically acceptable acid or base (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine.

As used herein, the phrase “cell proliferative disorder” refers to a neoplasm. That is an abnormal growth of cells or a growth of abnormal cells which reproduce faster than normal. A neoplasm creates an unstructured mass (a tumor) which can be either benign or malignant. The term “benign” refers to a tumor that is non-cancerous, e.g., its cells do not invade surrounding tissues or metastasize to distant sites. The term “malignant” refers to a tumor that is cancerous, and/or metastastic, i.e., invades contiguous tissue or is no longer under normal cellular growth control. Non-limiting examples of cell proliferative disorders that may be treated by the present invention include glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides. In preferred embodiments, cell proliferative disorder can be glioma, melanoma, lung cancer, lymphoma, colorectal and/or colon cancer, head and neck or ovarian cancer. In other preferred embodiments, the cell proliferative disorder is glioma or melanoma.

As used herein the term “standard dose intensity” of TMZ means a 5/28 dosing regimen, with a dosing schedule of 150-200 mg/m² of TMZ per day, administered for 5 days in a 28 day cycle for a maximal total dose of 1000 mg/m²/4 weeks. This dosing regimen provides a TMZ “dose intensity” of 1.0.

As used herein the term “enhanced dose intensity” of TMZ means a dosing regimen and/or dosing schedule which provides a dose intensity of TMZ, which is 1.2-4.2, preferably 1.4-2.8, more preferably 1.8-2.8 times more intense (compared with the standard dose intensity). See, U.S. Patent Application Publication No. US 2006/0100188, Tables 1 and 2 at pages 2 and 3 for illustrative dosing regimens using enhanced dosing intensities, the entirety of which is hereby incorporated by reference.

As used herein, the term “treating” is intended to mean mitigating or alleviating a cell proliferative disorder (e.g., glioma, etc.) in a mammal such as a human.

As used herein the term “capsule” refers to a special container or enclosure made of methyl cellulose, polyvinyl alcohols, or denatured gelatins or starch for holding or containing a composition comprising a formulation of the present invention and a carrier. Hard shell capsules are typically made of blends of relatively high gel strength bone and pork skin gelatins. The capsule itself may contain small amounts of dyes, opaquing agents, plasticizers, and preservatives.

As used herein the term “tablet” refers to a compressed or molded solid containing a composition comprising a formulation of the present invention and a carrier with suitable diluents. The tablet can be prepared by compression of mixtures or granulations obtained by wet granulation, dry granulation or by compaction.

As used herein the phrase “oral gel” refers to a composition comprising a formulation of the present invention and a carrier dispersed or solubilized in a hydrophilic semi-solid matrix.

As used herein the phrase “orally consumable film” refers to a composition comprising a formulation of the present invention and an edible film carrier.

As used herein the phrase “powders for constitution” refers to powder blends containing a composition comprising a formulation of the present invention and a carrier with suitable diluents which can be suspended in water or juices.

As used herein the term “diluent” refers to a substance that usually makes up the major portion of the composition. Suitable diluents include sugars such as lactose, sucrose, mannitol, and sorbitol; starches derived from wheat, corn rice, and potato; and celluloses such as microcrystalline cellulose. The amount of diluent in the composition can range from about 10% to about 90% by weight of the total composition, preferably from about 25% to about 75%, more preferably from about 30% to about 60% by weight, even more preferably from about 12% to about 60%.

As used herein the term “disintegrant” refers to a substance added to the composition to help it break apart (disintegrate) and release the medicinal agent(s). Suitable disintegrants include starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2% to about 15% by weight of the composition, more preferably from about 4% to about 10% by weight.

As used herein the term “binder” refers to a substance that binds or “glues” powders together and makes them cohesive by forming granules, thus serving as the “adhesive” in the composition. Binders add cohesive strength already available in the diluent or bulking agent. Suitable binders include sugars such as sucrose; starches derived from wheat, corn rice, and potato; natural gums such as acacia, gelatin, and tragacanth; derivatives of seaweed such as alginic acid, sodium alginate, and ammonium calcium alginate; cellulosic materials such as methylcellulose, sodium carboxymethylcellulose, and hydroxypropylmethylcellulose; polyvinylpyrrolidinone; and inorganics such as magnesium aluminum silicate. The amount of binder in the composition can range from about 2% to about 20% by weight of the composition, more preferably from about 3% to about 10% by weight, even more preferably from about 3% to about 6% by weight.

As used herein the term “lubricant” refers to a substance added to the composition to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols, and d'l-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

As used herein the term “glidant” refers to a substance that prevents caking and improves the flow characteristics of granulations, so that flow is smooth and uniform. Suitable glidants include silicon dioxide and talc. The amount of glidant in the composition can range from about 0.1% to about 5% by weight of the total composition, preferably from about 0.5% to about 2% by weight.

As used herein the phrase “coloring agent” refers to a substance that provides coloration to the composition. Such substances can include food grade dyes and food grade dyes adsorbed onto a suitable adsorbent such as clay or aluminum oxide. The amount of the coloring agent can vary from about 0.1% to about 5% by weight of the composition, preferably from about 0.1% to about 1%.

The present invention provides formulations, kits and methods comprising the use of TMZ or a pharmaceutically acceptable salt thereof in combination with a multi-kinase targeted kinase inhibitor.

The amount of TMZ to be administered and the frequency of administration are to be decided on a case by case basis by the attending physician.

In one embodiment, TMZ may be administered as an oral or intravenous dose in the range of about 150 to about 200 mg/m² per day for 5 days in a 28-day treatment cycle. In other embodiments, TMZ may also be administered at a dose of 100 mg/m² per day for 14 days in a 21 day cycle. In other embodiments, TMZ may be administered at a dose of 150 mg/m² for 7 days in a 14 day cycle. In other embodiments, TMZ may be administered at a dose of 100 mg/m² per day for 21 days in a 28 day cycle.

In one embodiment, the therapeutically effective amount of TMZ (or pharmaceutically acceptable salt thereof) is either a standard or enhanced dose intensity of TMZ based upon the methylation state of the O⁶-methylguanine-DNA methyltransferase (MGMT) gene in a sample obtained from the patient. If the gene (e.g., the promoter region) encoding MGMT in a sample from the patient is methylated, a standard dose intensity of TMZ is administered; however, if the gene encoding MGMT is not methylated (i.e., below the level of detection), an enhanced dose intensity of TMZ is administered to the patient. See U.S. Patent Publication No. 2006/0100188, in particular, exemplary enhanced dose intensities for TMZ are provided in Tables 1 and 2; methods to assess whether or not the MGMT gene is methylated are provided on pages 15-20; and the term “sample” is defined on page 13. The disclosure of U.S. 2006/0100188 is incorporated by reference herein.

TMZ may be administered by any suitable route. In a preferred embodiment TMZ is to be administered orally. In another preferred embodiment, TMZ is to be administered intravenously.

The multi-targeted kinase inhibitor could be any multi-targeted kinase inhibitor. Exemplary multi-targeted kinase inhibitors are described in the art. Examples of multi-targeted kinase inhibitors include Sutent® (sunitinib; SU11248) from Pfizer, Nexavar® (sorafenib; Bay 43-9006) from Onyx Pharmaceuticals; dasatinib (BMS-354825) from Bristol-Myers Squibb; Zactima® (ZD6474) from Astra Zeneca; Tykerb® (lapatinib) from Glaxo Smith Kline; STI571 from Novartis; AMG 706 from Amgen; MP-412 from Aveo Pharmaceuticals; CEP-701 (lestaurtinib) from Cephalon; XL647 from Exelixis; XL999 from Exelixis; MLN518 (formerly known as CT53518) from Millennium Pharmaceuticals; PKC412 from Novartis; AMN107 from Novarits; AEE 788 from Novartis; OSI-930 from OSI Pharmaceuticals; OSI-817 from OSI Pharmaceuticals; axitinib (AG-013736) from Pfizer; ARRY-334543 from Array BioPharma; MG-90265 from MethylGene, Inc.; and AZD6244 (ARRY-142886), or a pharmaceutically acceptable salt of any of these agents, or a combination of two or more of these agents. In a preferred embodiment, the multi-targeted kinase inhibitor is sunitinib, or a pharmaceutically acceptable salt thereof. In another preferred embodiment, the multi-targeted kinase inhibitor is sorafenib or a pharmaceutically acceptable salt thereof.

The multi-targeted kinase inhibitor could also be a bi-specific antibody (or an antigen binding fragment thereof) which inhibits two or more kinase. Antibodies which inhibit kinases are well known in the art. Bi-specific antibodies that bind and inhibit kinases can made using only routine experimentation.

Generally, an amount of multi-targeted kinase inhibitor to be administered in combination with TMZ is decided on a case by case basis by the attending physician. As a guideline, the extent of the cell proliferative disorder, the body weight, and the age of the patient will be considered, among other factors, when setting an appropriate dose. As noted above, the above amounts may vary on a case-by-case basis. In some embodiments, TMZ and the multi-targeted kinase inhibitor may be administered in combination with other agents or compounds, including, but not limited to PARP inhibitors, an O⁶-alkylguanine-DNA-alkyltransferase (ATase) inhibitor (e.g., O⁶BG), an anti-emetic agent, a farnesyl protein transferase inhibitor or another anti-neoplastic agent.

In one embodiment, the formulations and kits of the present invention are for oral administration. For oral preparations, a pharmaceutically acceptable carrier (which includes diluents, excipients, or carrier materials) is also present in the formulation. The carrier is suitably selected with respect to the intended form of administration, i.e., oral tablets, capsules (either solid-filled, semi-solid filled, or liquid filled), powders for constitution, oral gels, orally consumable films, elixirs, syrups, suspensions, and the like, and consistent with conventional pharmaceutical practices. For example, for oral administration in the form of tablets or capsules, the pharmaceutically active agents may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms), and the like. Moreover, when desired or needed, suitable binders, lubricants, disintegrants, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol, and waxes. Suitable lubricants include boric acid, sodium benzoate, sodium acetate, sodium chloride, and the like. Suitable disintegrants include starch, methylcellulose, guar gum, and the like. Suitable disinfectants include benzalkonium chloride and the like. Sweetening and flavoring agents and preservatives may also be included where appropriate.

Additionally, the formulations and kits of the present invention may be formulated in sustained release form to provide the rate controlled release of any one or more of the pharmaceutically active agents to optimize the therapeutic effects. Suitable compositions for sustained release include layered tablets (e.g., containing layers of varying disintegration rates or controlled release polymeric matrices impregnated with the medicinal agents) that are shaped in tablet form or capsules containing such impregnated or encapsulated porous polymeric matrices.

Conventional methods for preparing tablets are known. Such methods include dry methods such as direct compression and compression of granulation produced by compaction, wet methods, or other special procedures.

In another embodiment, the formulations and kits of the present invention are for parenteral administration, for example, intravenous, intratumoral, subcutaneous, or intramuscular administration.

Thus, to prepare an aqueous solution for parenteral injection, it is possible to use a co-solvent, e.g., an alcohol such as ethanol or a glycol such as polyethylene glycol or propylene glycol, or glycerin, and optionally, a hydrophilic surfactant such as Tween® 80. An oily solution injectable intramuscularly can be prepared, e.g., by solubilizing the active principle with a triglyceride or a glycerol ester. The substantially non-aqueous carrier (excipient) can be any substance that is biocompatible and liquid or soft enough at body temperature. The carrier is usually hydrophobic and commonly organic, e.g., an oil or fat of vegetable, animal, mineral or synthetic origin or derivation. Preferably, but not necessarily, the carrier includes at least one chemical moiety of the kind that typifies “fatty” compounds, e.g., fatty acids, alcohols, esters, etc., i.e., a hydrocarbon chain, an ester linkage, or both. “Fatty” acids in this context include acetic, propionic and butyric acids, through straight- or branched-chain organic acids containing up to 30 or more carbon atoms.

Preferably, the carrier is immiscible in water and/or soluble in the substances commonly known as fat solvents. The carrier can correspond to a reaction product of such a “fatty” compound or compounds with a hydroxy compound, e.g., a mono-hydric, di-hydric, trihydric or other polyhydric alcohol, e.g., glycerol, propanediol, lauryl alcohol, polyethylene or -propylene glycol, etc. These compounds include the fat-soluble vitamins, e.g., tocopherols and their esters, e.g., acetates sometimes produced to stabilize tocopherols. Sometimes, for economic reasons, the carrier may preferably comprise a natural, unmodified vegetable oil such as sesame oil, soybean oil, peanut oil, palm oil, or an unmodified fat. Alternatively the vegetable oil or fat may be modified by hydrogenation or other chemical means which is compatible with the present invention. The appropriate use of hydrophobic substances prepared by synthetic means is also envisioned.

Pharmaceutical compositions suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g., human serum albumin), toxicity agents (e.g., NaCl), preservatives (e.g., thimerosol, cresol or benzylalcohol), and surfactants (e.g., Tween or polysorabates) in sterile water for injection.

Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized powder in a separate compartment.

The following human glioblastoma xenograft model may be employed to ascertain the efficacy of the formulations and methods described herein. Human glioblastoma cell U87MG are inoculated subcutaneously into female nude mice (age 4-6 wks). Xenograft tumor growth is followed by measuring the tumor size using a caliper. Once tumor size reaches about 100 mm³ (average), mice carrying the xenograft tumors are grouped and treated with different doses of the combination of TMZ and a multi-targeted kinase inhibitor. Likewise, human glioblastoma cell U373 may be used to establish a xenograft model.

### Example

### Efficacy of TMZ and/or Sunitinib in U87MG Glioblastoma Xenografts

The combination of TMZ and a multi-targeted kinase inhibitor (e.g., sunitinib) for treating a cell proliferative disorder was examined using a human glioblastoma xenograft model. In particular, the glioblastoma model U87MG was used to evaluate the efficacy of multi-targeted kinase inhibitor sunitinib as a single agent compared to TMZ (Temozolomide, Temodar®), a chemotherapeutic drug clinically approved for treating brain tumor, as a single agent. In addition, this study looked at the efficacy of the combination of TMZ with sunitinib in comparison to the efficacy with either agent alone.

In brief, nude mice were inoculated with U87-MG glioblastoma cells and the resultant tumors treated with control, or various concentrations of TMZ, sunitinib, or a combination of both TMZ and sunitinib. More specifically, female nude mice (strain NU/NU), aged 6-8 weeks old were purchased from Charles River Laboratory. Five million glioblastoma U87-MG were mixed 1:1 (volume:volume) with Matrigel (Cat. # 354234, BD Biosciences) on ice and mixtures were inoculated subcutaneously to the flank of each mouse. Dosing was initiated on day 8 after inoculation when the tumor size was approximately 100 mm³. TMZ was administered qd, ip for five consecutive days. Sunitinib was administered qd, po for 9-12 days consecutive days (until the end of the studies). The dosing volume was approximately 0.1 ml. Tumor size and body weight was measured two to three times per week.

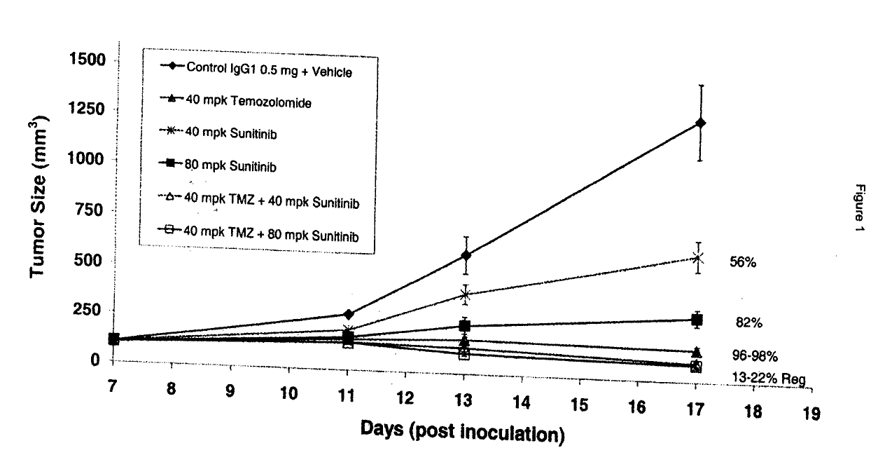

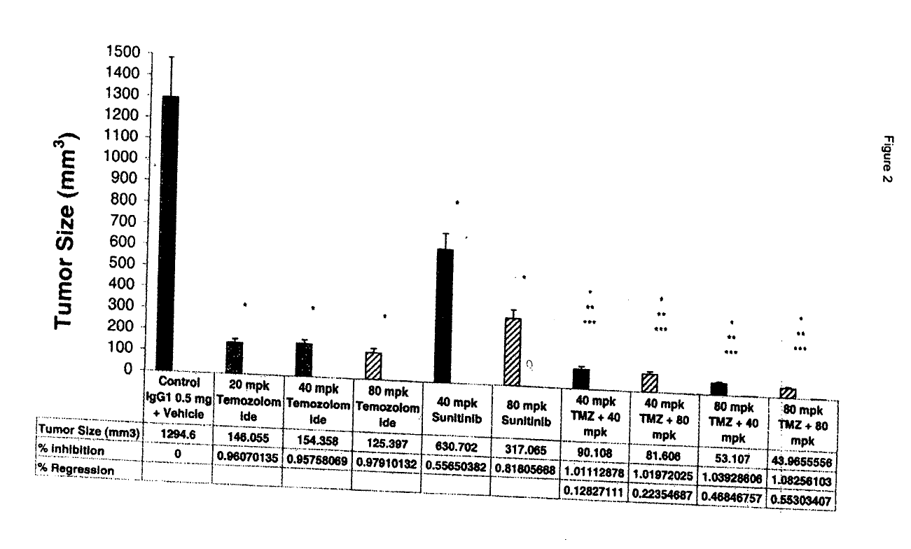

Table 1 displays 10 different dosing regimens used for the experiments shown in FIGS. 1 and 2. There were 10 nude mice per dosing regimen.

TABLE 1

| Dosing | | |
|---|---|---|
| Regimen | Dose 1 (qd, ip) | Dose 2 (qd, po) |
| 1 | Saline with 10% DMSO | 20% HPBCD |
| 2 | 20 mpk TMZ | — |
| 3 | 40 mpk TMZ | — |
| 4 | 80 mpk TMZ | — |
| 5 | — | 40 mpk sunitinib malate |
| 6 | — | 80 mpk sunitinib malate |
| 7 | 40 mpk TMZ | 40 mpk sunitinib malate |
| 8 | 40 mpk TMZ | 80 mpk sunitinib malate |
| 9 | 80 mpk TMZ | 40 mpk sunitinib malate |
| 10 | 40 mpk TMZ | 80 mpk sunitinib malate |

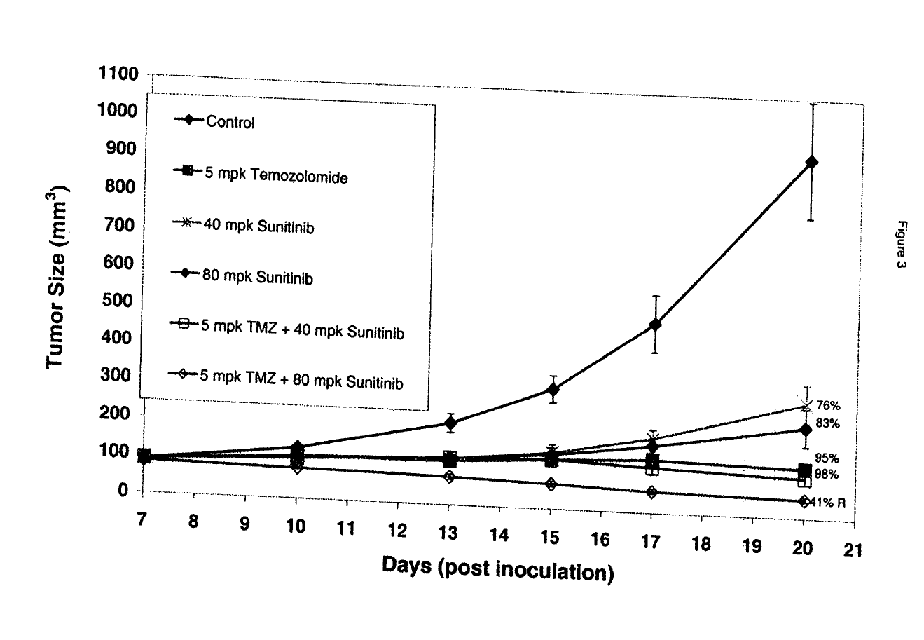

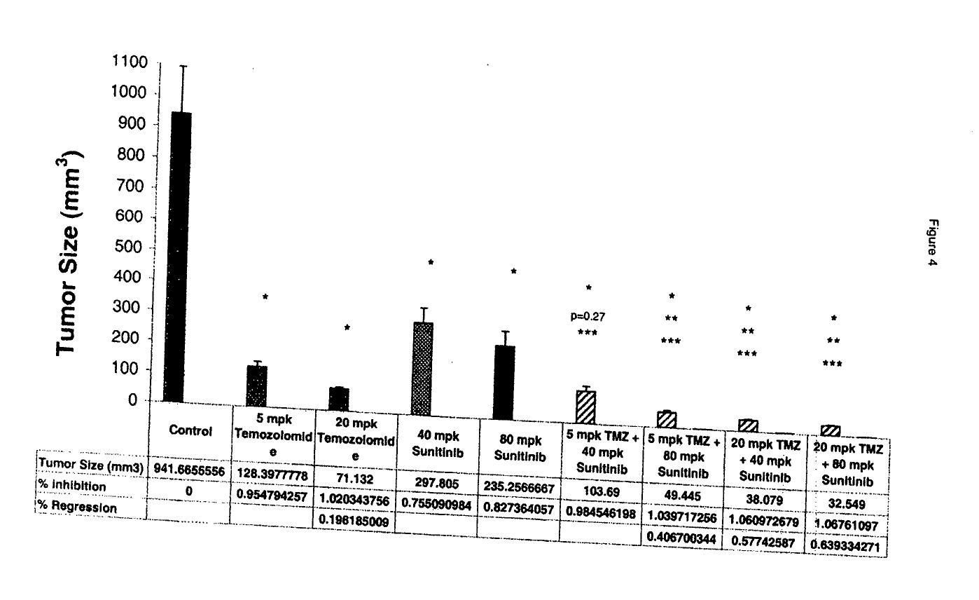

Table 2 displays 9 different dosing regimens used for the experiments shown in FIGS. 3 and 4. There were 10 nude mice per dosing regimen.

TABLE 2

| Dosing | | |
|---|---|---|
| Regimen | Dose 1 (qd, ip) | Dose 2 (qd, po) |
| 1 | Saline with 10% DMSO | 20% HPBCD |
| 2 | 5 mpk TMZ | — |
| 3 | 20 mpk TMZ | — |
| 4 | — | 40 mpk sunitinib malate |
| 5 | — | 80 mpk sunitinib malate |
| 6 | 5 mpk TMZ | 40 mpk sunitinib malate |
| 7 | 5 mpk TMZ | 80 mpk sunitinib malate |
| 8 | 20 mpk TMZ | 40 mpk sunitinib malate |
| 9 | 20 mpk TMZ | 80 mpk sunitinib malate |

FIG. 1 illustrates mean tumor growth curves of U87MG (glioblastoma) xenografts tumors treated with TMZ alone or in combination with sunitinib according to the dosing regimens described in Table 1. It shows that TMZ is more effective than Sunitinib at inhibiting the growth of U87MG glioblastoma xenografts, while TMZ in combination with sunitinib is more effective than either TMZ or sunitinib alone at decreasing tumor growth in U87MG glioblastoma xenografts.

FIG. 2 illustrates U87MG xenograft tumor size on Day 17 post-inoculation in nude mice receiving the dosing regimens shown in Table 1. On Day 17 post inoculation (i.e., day 9 post dosing initiation), TMZ alone inhibited the U87MG glioblastoma xenograft tumor growth by 96%, 96% and 98%, at 20, 30 and 40 mpk dose levels, respectively (see FIG. 2). In contrast, multi-targeted kinase inhibitor sunitinib inhibited tumor growth in the same model only by 56% and 82% at 40 and 80 mpk dose levels, respectively (see FIG. 2). Moreover, TMZ in combination with sunitinib was more effective than either TMZ or sunitinib used alone. In particular, 101%, 102%, 104% or 108% inhibition of tumor growth was observed using different dosing regimens including a combination of TMZ and sunitinib. Notably, the highest combination dose (i.e., 80 mpk TMZ plus 40 mpk sunitinib) resulted in 47% tumor regression (compared to its initial size when dosing initiated on day 8 post inoculation). It should also be noted that mice tolerated these dosage regimens of TMZ and sunitinib well, exhibiting no more than 5% body weight loss.

FIG. 3 illustrates mean tumor growth curves of U87MG xenografts tumors treated with TMZ alone or in combination with sunitinib, according to the dosing regimens shown in Table 2. It shows that TMZ is more effective than Sunitinib at inhibiting the growth of U87MG glioblastoma xenografts, while TMZ in combination with sunitinib is more effective than either TMZ or sunitinib alone at decreasing tumor growth in U87MG glioblastoma xenografts.

FIG. 4 illustrates U87MG xenograft tumor size on Day 20 post-inoculation in nude mice receiving the dosing regimens described in Table 2. On Day 20 post inoculation (i.e., day 12 post dosing initiation), TMZ alone inhibited the U87MG glioblastoma xenograft tumor growth by 95% and 102% at 5 and 20 mpk dose levels, respectively (see FIG. 4). In contrast, multi-targeted kinase inhibitor sunitinib inhibited tumor growth in the same model only by 76% and 83% at 40 and 80 mpk dose levels, respectively (see FIG. 4). TMZ in combination with sunitinib was more effective than either TMZ or sunitinib alone. In particular, 98%, 104% 106% or 107% inhibition of tumor growth was observed using different combination dosing regimens of TMZ and sunitinib. Notably, the combination doses of TMZ and sunitinib result in 41%, 58% and 64% tumor regression (compared to its initial size when dosing initiated on day 8 post inoculation). It should also be noted that mice tolerated these dosage regimens of TMZ and sunitinib well, exhibiting no more than 5% body weight loss.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1.-4. (canceled)

5. A method for treating a patient having a cell proliferative disorder comprising administering to the patient a therapeutically effective amount of temozolomide (TMZ) or a pharmaceutically acceptable salt thereof and a therapeutically effective amount of sunitinib or a pharmaceutically acceptable salt thereof.

6. The method of claim 5, wherein the cell proliferative disorder is a brain tumor.

7. The method of claim 5, wherein the brain tumor is a glioma.

8. The method of claim 5, wherein the cell proliferative disorder is melanoma.

9. A formulation comprising a therapeutically effective amount of temozolomide (TMZ) or a pharmaceutically acceptable salt thereof and a therapeutically effective amount of a sunitinib or a pharmaceutically acceptable salt thereof.

10.-12. (canceled)

13. A kit comprising:
a) a first container having a therapeutically effective amount of TMZ or a pharmaceutically acceptable salt thereof;
b) a second container having a therapeutically effective amount of a sunitinib or a pharmaceutically acceptable salt thereof; and
c) instructions for use to treat a cell proliferative disorder.

14. The kit of claim 13, wherein the cell proliferative disorder is a brain tumor.

15. The kit of claim 13, wherein the brain tumor is a glioma.

16. The kit of claim 13, wherein the cell proliferative disorder is a melanoma.

17.-19. (canceled)
